(19) United States
(12) Patent Application Publication
(10) Pub. No.: US 2021/0393549 A1
(43) Pub. Date: Dec. 23, 2021

(54) New use of R-enantiomer of adrenergic beta 2 receptor agonists for treatment of inflammatory bowel disease and its extra intestinal manifestations

(71) Applicant: Tan WEN, Guangdong (CN)

(72) Inventor: Tan WEN, Guangdong (CN)

(21) Appl. No.: 17/274,175

(22) PCT Filed: Sep. 7, 2019
(86) PCT No.: PCT/US2019/050120

§ 371 Date: Mar. 7, 2021

Related U.S. Application Data
(60) Provisional application No. 62/728,800, filed on Sep. 9, 2018.

(51) Int. Cl. A61K 31/137; A61K 31/27; A61K 31/138; A61K 31/538; A61K 31/4704; A61K 31/55; A61K 31/655; A61K 31/573; A61K 31/407; A61K 31/545; A61K 31/427; A61K 31/43; A61K 31/345; A61K 31/65; A61K 31/421; A61K 31/397; A61K 31/4439; A61K 39/395; A61P 37/06; A61P 17/06

(52) U.S. Cl. CPC A61K 31/137; A61P 17/06; A61K 31/138; A61K 31/538; A61K 31/4704; A61K 31/55; A61K 31/655; A61K 31/573; A61K 31/407; A61K 31/545; A61K 31/427; A61K 31/43; A61K 31/345; A61K 31/65; A61K 31/421; A61K 31/397; A61K 31/4439; A61K 39/3955; A61P 37/06; A61K 31/27

(57) ABSTRACT

This invention disclosed a new use of optically pure R-enantiomer of adrenergic β2 agonists including R-salbutamol, R-terbutaline, R-clenbuterol and R-bambuterol for treatment of inflammatory bowel disease and its extra intestinal manifestations including skin diseases.

### FIELD OF INVENTION

This invention involved new uses of optically pure R-enantiomer of adrenergic β2 receptor agonist for treatment of inflammatory bowel disease (IBD) and the extra intestinal manifestations of inflammatory bowel diseases. The exact cause of IBD is unknown, but IBD is the result of a defective immune system. IBD includes Crohn's disease and ulcerative colitis, that are characterized by chronic inflammation of the gastrointestinal tract, persistent diarrhea abdominal pain rectal bloody stools, weight loss and fatigue. IBD is associated with extra-intestinal manifestations such as liver problems, arthritis, skin manifestations and eye problems. Currently there is no effective therapy for curing of IBD. The main treatments for symptoms control of IBD involve anti-inflammation drugs such as 5-aminosalicylate and corticosteroids, and immunosuppression agents such as cyclosporine. Long term uses of these drugs often induce serious adverse effects. Surgery sometimes is needed for IBD. Therefore, there is unmet medical need for a more effective and safer medicine for treatment of IBD to a patient in need.

This invention disclosed a new use of R-enantiomer of chiral adrenergic β2 receptor agonists for treatment of IBD and the extra intestinal manifestation of IBD. This invention also provided that S-enantiomer of chiral adrenergic β2 receptor agonists cause unexpectedly an exacerbation of IBD. Therefore, the R-enantiomer of β2 receptor agonists is superior to its S-enantiomer or its racemic form for treatment of IBD to a patient in need. This invention disclosed new used of R-enantiomers of adrenergic β2 receptor agonists for treatment of IBD and its extra intestinal manifestations with reduced adverse effects in comparison of its racemic and other marketed drugs. The R-enantiomers of adrenergic β2 receptor agonists according to this invention include short acting β2 agonists (SABA), preferably R-salbutamol, and long acting β2 agonists (LABA), preferably R-bambuterol and R-clenbuterol. This invention also disclosed a combination use of at least one of R-enantiomers of adrenergic β2 receptor agonists with at least one of anti inflammation medicine such as corticosteroids and 5-aminosalicylates or with at least one of immunosuppressing agents, or one of antibiotics as well as with one of antibodies and one SIP receptor modulators used for IBD. This invention disclosed a new used of R-enantiomers of adrenergic β2 receptor agonist for treatment of extra intestinal manifestation of IBD, including arthritis, uveitis, rhinitis, ankylosing spondylitis. vitiligo, psoriasis, amyloidosis, eczema, acne, and primary sclerosing cholangitis.

### BACKGROUND OF INVENTION

Inflammatory bowel disease (IBD) is a term for two conditions (Crohn's disease and ulcerative colitis) that are characterized by chronic inflammation of the gastrointestinal tract. Prolonged inflammation results in damage to the GI tract. Some common symptoms are: persistent diarrhea, abdominal pain, rectal bleeding, bloody stools, weight loss and Fatigue. (Baumgart D C, & Carding S R Inflammatory bowel disease: cause and immunobiology. Lancet. 369 (9573): 1627, May 2007). Associated complaints or diseases include arthritis, pyoderma gangrenosum, primary sclerosing cholangitis, and non-thyroidal illness syndrome (NTIS) (Liu S, et al., “Nonthyroidal illness syndrome: is it far away from Crohn's disease?. Journal of Clinical Gastroenterology. 47 (2): 153-9. February 2013). Lastly, Crohn's disease and ulcerative colitis present with extra-intestinal manifestations (such as liver problems, arthritis, skin manifestations and eye problems) in different proportions. (Extraintestinal manifestations of inflammatory bowel disease, Levine J S et. al Gastroenterology & hepatology Vol.&, Issue 4, 235, April 2011)

The overall prevalence for IBD is 396 cases per 100,000 persons annually. IBD resulted in a global total of 51,000 deaths in 2013. The increased incidence of IBD since World War II has been linked to the increase in meat consumption worldwide, supporting the claim that animal protein intake is associated with IBD. Inflammatory bowel diseases are increasing in Europe. The exact cause of IBD is unknown, but IBD is the result of a defective immune system. A properly functioning immune system attacks foreign organisms, such as viruses and bacteria, to protect the body. In IBD, the immune system responds incorrectly to environmental triggers, which causes inflammation of the gastrointestinal tract. There also appears to be a genetic component.

Several types of medications have been used to treat IBD: 5-aminosalicylates, corticosteroids (such as prednisone), immune-modulators and the newest class approved for IBD: the biologics. Several vaccinations for patients with IBD are recommended to prevent infections. Severe IBD may require surgery to remove damaged portions of the gastrointestinal tract, but advances in treatment with medications means that surgery is less common than it was a few decades ago. Since Crohn's disease and ulcerative colitis affect different parts of the GI tract, the surgical procedures are different for the two conditions.

Steroids, such as the glucocorticoid prednisone, are frequently used to control disease flares and were once acceptable as a maintenance drug. Biological therapy for inflammatory bowel disease, especially the TNF inhibitors, is used in people with more severe or resistant Crohn's disease and sometimes in ulcerative colitis.

Despite recent advances in treatments, there remains a need for a safe, well-tolerated therapy with a rapid onset, and increased capacity for maintaining long-term remission. In addition, long-term use of drugs such as steroid can induce serious side effects. Therefore, there is a urgent need for better medicine for IBD with better efficacy and less adverse effects and lower cost.

### DETAIL OF INVENTION

Animal models of IBD are valuable and indispensable tools that provide a wide range of options for investigating involvement of various factors into the pathogenesis of IBD and to evaluate different therapeutic options. Even if none of the individual models represents all aspects and stages i.e. of ulcerative colitis mostly restricted to the colon mucosa or of Crohn's disease affecting the whole gastrointestinal tract with trans-mural inflammation (R. B. Sartor and M. Muehlbauer, “Microbial host interactions in IBD: implications for pathogenesis and therapy,” Current Gastroenterology Reports, vol. 9 (6), pp. 497, 2007)

The dextran sulphate sodium (DSS) induced colitis model is well appreciated and widely used model of inflammatory bowel disease. The DSS-induced colitis model has some advantages when compared to other animal models of colitis. For example, an acute, chronic, or relapsing model can be produced easily by changing the concentration of administration of DSS. Moreover, dysplasia that resembles the clinical course of human UC occurs frequently in the chronic phase of DSS-induced colitis. DSS-induced model for studying colitis-associated carcinogenesis has been recently reviewed. Furthermore, studies that validated DSS model by using different therapeutic agents for human IBD showed that DSS-induced colitis can be used as a relevant model for the translation of mice data to human disease. (Perse M & Cerar A, Dextran sodium sulphate colitis mouse model: traps and tricks. J of Biomedicine and Biotechnology, Vol. 2012, ID 18617, March 2012). In this invention, DSS mouse model was used. In prior art, R-salbutamol has been shown to be effective in treatment of asthma. It was also used as a topic skin cream for treatment of skin disease such as discoid lupus. (Jemec G B et. al. m A randomized controlled trial of R-salbutamol for topical treatment of discoid lupus erythematosus. Br. J. Demortol. 161(16):1365, December 2009) and for facilitating chronic wound healing in skin. However, IBD is a disease completely differs than asthma and discoid lupus (connective tissue disease) or impaired chronic wound healing of skin. The above clinic manifestations and histological features of colitis were significantly ameliorated after treatments with R-salbutamol which was added into the DSS drinking water at the day one. The DAI of IBD were significantly improved. There were increased body weight, reduced diarrhea and fecal occult blood in IBD animals. In pathology and histological examination, the shortening of large bowel was reduced after treatment of R-salbutamol. There was few aberrant or erosion of mucosa. The inflammation responses of mucosa and submucosa were significant alleviated. A significant number of newly grown crypts was appeared. There was a clear recovery of epithelial lining of colon mucosa. The lamina propria remained intact and no visible inflammatory infiltration. The histological score in R-salbutamol treated animals were significantly reduced in comparison of DSS induced colitis mice. This invention disclosed that R-salbutamol can be used for prevent and treatment of IBD. This disclosure is novel and cannot be anticipated by a person in art. IBD usually cannot be treated via skin topically. IBD is chronic disease and has different mechanism and clinic manifestations than those diseases, which can be treated with R-salbutamol or other β2 agonists, noted above or disclosed in prior art. Whether R-salbutamol or other similar β2 agonist could be used for treatment of IBD has nerve been reported in prior art.

This invention disclosed that R-salbutamol can be used for prevent or treatment of IBD. In one embodiment, IBD was induced in mice by add DSS into drink water for 7 days. The clinical manifestation of colitis was seen days after and the disease activity index, which including weight loss, diarrhea, fecal occult blood (FOB), were increased. (Cooper, S. et al., Clinic pathologic study of dextran sulfate sodium experimental murine colitis, Laboratory Investigation, vol. 69, no. 2, p. 238-249, 1993)

The mice were scarified at day 10th for anatomy and histology evaluations. The length of colon-rector was shortened. The histology of inflammatory features of acute colitis was found in large bowel, which includes mucin depletion, epithelial degeneration, and necrosis leading to disappearance of epithelial cells. This was accompanied by neutrophils infiltration and destruction of lamina propria and submucosa, cryptitis, (trans-epithelial migration of neutrophils into mucosal epithelium), and phlegmonous inflammation in mucosa and submucosa. There were also erosion and phlegmonous inflammation of mucosa and submucosa and disappearance of crypts. Each of the above histological features was scored individually and added together which is then collectively expressed as “histological score”, for quantitative evaluation (Cooper, S. et al., above).

The above clinic manifestations and histological features of colitis were significantly ameliorated after treatments with R-salbutamol which was added into the DSS drinking water at the day one. The DAI of IBD were significantly improved. There were increased body weight, reduced diarrhea and FOB. In pathology and histological examination, the shortening of large bowel was reduced after treatment of R-salbutamol. There was also few aberrant or erosion of mucosa. The inflammation responses of mucosa and submucosa were significant alleviated. A significant number of newly grown crypts was appeared along with clear recovery of epithelial lining of colon mucosa. The lamina propria remained intact and no visible inflammatory infiltration. The histological score in R-salbutamol treated animal were significantly reduced in comparison of DSS induced colitis mice. This invention disclosed that R-salbutamol can be used for prevent and treatment of IBD. This disclosure is novel and it cannot be anticipated by a person in art.

In other embodiment, this invention provided that other short-acting R-enantiomers of β2 agonist including R-terbutaline and long-lasting β2 agonist including R-bambuterol and R-clenbuterol also demonstrated the similar effects in prevent and treatment of DSS induced IBD as did by R-salbutamol. This invention disclosed a new used of R enantiomer or R′R-enantiomer of adrenergic β2 agonists in prevent and treatment of IBD. The disclosed of this invention is novel and cannot be anticipated by a person in art.

A common used therapy for IBD is corticosteroids. In one embodiment, this invention provided that R-enantiomer adrenergic agonists had superior efficacy in ameliorating of IBD in comparing dexamethasone.

Currently, most of chiral adrenergic β2 agonists are marketed and used as racemic form which is a mixture of R and S enantiomers. It is known that one of the opposite enantiomers sometimes may be inert and do not have same biological activity as the other. However, there is no report about the toxic effects of S-enantiomer on IBD in prior art. In one embodiment, instead of R-salbutamol, S-salbutamol was used in IBD mice in the same fashion as R-salbutamol. Surprisingly, S-salbutamol showed no amelioration or therapeutic effects at all as did by R-salbutamol, instead, S-salbutamol worsened significantly the clinic manifestation and inflammatory features in mice in response to DSS stimulation. There DAI were significantly exacerbated in comparing untreated DSS mice. In particular, the FOB was significantly worsened than untreated DSS mice. The length of colon-rector was further shortened than untreated DSS mice. The erosion of mucosa, the damage of epithelial cells and loss of crypts were much more severe in comparison of untreated DSS mice. There was enhancement in inflammatory infiltrations in mucosa and submucosa, lamina propria and sometime seen in muscular layer, as well as more severe destruction in lamina propria in comparing untreated DDS mice. The histological score was significantly higher than untreated DSS mice. This invention disclosed that S-salbutamol was toxic to the bowel, and it can further exacerbate the clinic symptoms and inflammatory reactions in IBD. Furthermore, to remove S-enantiomer salbutamol from racemic salbutamol, e.g. to use R-salbutamol instead of racemic salbutamol can reduce the toxicity and unwanted adverse effects caused by S-salbutamol. This disclosure of this invention should be considered as novel. These toxic effects of S-enantiomer β2 agonists were unexpected and cannot be anticipated by a person in art.

In other embodiment, other S-enantiomers of β2 adrenergic agonist including S-terbutaline and S-bambuterol were used in the same fashion as S-salbutamol in DSS mice. Similar toxic effects were observed. Both disease active index (DAI) and histological score in S-enantiomer β2 agonists treated DSS mice were much worsened than untreated DSS mice. This invention disclosed that S-enantiomer β2 agonists were toxic to the bowel, and it can further exacerbate the clinic symptoms and inflammatory reactions in IBD. Furthermore, this invention disclosed a new use of R-enantiomer β2 agonists in treatment of IBD with reduced toxicity. This invention provided that to remove S-enantiomer from racemic e.g. to use R-enantiomer β2 agonists instead of racemic β2 agonists can reduce the toxicity and adverse effects caused by S-enantiomers. This disclosure by this invention should be considered as novel. These toxic effects were unexpected and cannot be anticipated by a person in art.

This invention also disclosed the used of R-salbutamol for treatment of sub acute or chronic phases of IBD. In one embodiment, IBD was induced by DSS as mentioned above, along with the treatment of R-salbutamol, at day 7^{th}, DSS was removed and replaced with drinking water for additional 2 weeks. The disease active index (DAI) of mice was examined and evaluated periodically. The mice were scarified for pathological and histological evaluations at day of 23^{rd}. In DSS induce IBD mice, there were significant loss of weight and fecal occult blood at day 14^{th} and 22^{nd}. The overall condition of colitis was severe although there was slightly improvement comparing the day 7^{th}. The colon-rector length was significant shortened, and histological score were significant higher from control. In mice with DSS induced colitis, there were significant mononuclear leucocytes infiltration, few newly grew crypts with architectural disarray, denegation of epithelia, mucosa aberrant. There was also deep mucosal lymphocytosis, destruction of lamina propria and sometimes, transmural inflammation.

The disease active index (DAI) was significant improved after the R-salbutamol treatment in comparing untreated DSS animals. The body weights were similar as control normal mice; the fecal occult blood was significantly reduced after the treatment of R-salbutamol in comparing untreated DSS mice. The histological index was also significantly ameliorated after R-salbutamol treatment. There were almost completed regeneration and normal arranged crypts. The normal grew epithelia cell formed a completed surface of mucosa. The lamina propria was intact and appeared normal. There were few inflammatory infiltrations in either mucosa or submucosa.

In one embodiment, similar studied was done as above except the treatments started at day 7^{th} till day 22^{nd}, instead of at day 1^{st} till day 22^{nd} as did above. The results were similar as above and R-salbutamol showed significant therapeutic effects in DSS induced IBD mice.

This invention disclosed that R-salbutamol can be used for prevent or treatment of sub-acute or chronic IBD. This disclosure in this invention cannot be anticipated by a person in art.

In one embodiment, this invention further disclosed that (R)-salbutamol and other R-enantiomer β2 agonists had significant therapeutic effects in treating TNBS-induced colitis (a common used animal model for Crohn's disease) in mice. The effects of R-salbutamol were superior than infliximab, a biological drug for IBD. Male mice colitis was induced by rectal co-administration of ethanol and TNBS (Trinitrobenzenesulfonic acid). Severe colitis in mice that was characterized by weight loss and diarrhea. Histologically there were loss of normal structures, visible discrete epithelioid granuloma, multiple erosive lesions, massive transmural inflammation infiltration of lymphocytes, and the loss of goblet cells. However, the disease condition was great ameliorated after oral administration of R-salbutamol or R-clenbuterol. No significant different were found in weight loss, diarrhea, colon length between R-salbutamol or R-clenbuterol treated and normal control mice. There was significant alleviation of lesions, few inflammatory infiltration with growing crypts and recovery mucosa epithelia along the column. In addition, treatments with infliximab had similar therapeutic effects as did R-salbutamol, However, there were significant submucosa fibrosis and narrowing in lumen in intestine in IFX treated animals, which were not seen in R-salbutamol or R-clenbuterol treated animals. In addition, in TNBS treated colitis mice, there were perianal symptoms: erythema, abscesses and ulcers found in some mice, which may lead to perianal fissures or fistulas. However, these perianal manifestations in TNBS treated mice was significantly alleviated after treatment of R-salbutamol or R-bambuterol. The therapeutically effects of R-salbuterol, R-clenbuterol and R-bambuterol on the symptoms in TNBS induced Crohn's disease has never been reported in prior art.

These perianal pathological changes somewhat similar to hemorrhoid inflammation symptoms. In one embodiment, this invention disclosed that R-salbuterol, R-clenbuterol and R-bambuterol significantly alleviated hemorrhoid inflammation symptoms by given either orally or topically. This new used of beta2 agonists in treatment of hemorrhoid inflammation symptoms has never been disclosed in prior art.

In another embodiment, his invention disclosed that, in TNBS induced Crohn's disease, there were submucosa fibrosis and lumen narrowing which may lead to surgery in IBD patients. R-salbutamol treatment can significantly reduce the fibrosis and the narrowing of intestinal lumen. On the other hand, treatment with infliximab enhanced the submucosa fibrosis and lumen narrowing. These beneficial effects of R-salbutamol are novel and cannot be anticipated by a person in art.

In other embodiment, other R-enantiomers including short-acting β2 agonist, R-terbutaline or long-lasting β2 agonist R-bambuterol and R-clenbuterol were administered individually in sub-acute or chronic DSS mice as above. R-enantiomer β2 agonists. These drugs significantly ameliorated the sub-acute orchronic clinic symptoms and the pathological changes induced by DSS or TNBS. Both the disease active index and histologic score were significantly improved after treatment of each individual R-enantiomers of β2 agonist in comparison of untreated DSS or TNBS induced IBD mice. This invention disclosed that R-enantiomer β2 agonists can be used for treatment of sub-acute or chronic IBD including UC and Crohn's disease and having significant better therapeutic effects than marketed drugs. this disclosure of this invention cannot be anticipated by a person in art.

This invention further disclosed a new use of a group of R-enantiomers β2 agonists in prevent and treatment of IBD and related hemorrhoid symptoms mentioned above as following: short-acting β2 agonists: bitolterol, fenoterol, isoprenaline, orciprenaline or metaproterenol pirbuterol, procaterol, ritodrine; and Long-acting β2 agonists: arformoterol, formoterol, perforomist, salmeterol, trantinterol; and ultra-long-acting β2 agonists: abediterol, carmoterol, indacaterol, olodaterol, vilanterol, isoxsuprine, mabuterol and zilpaterol

In one embodiment, S-salbutamol and S-bambuterol were used for 8 days in the same way as did R-salbutamol mentioned above after IBD induced by DSS. Both DAI and histological score were examined periodically during 22 days. By surprise, both S-salbutamol and S-bambuterol showed no signs of amelioration or therapeutic effects as did by R-salbutamol, Instead, both S-salbutamol and S bambuterol worsened the disease manifestations and inflammatory response in comparing to DSS mice. The DAI was much worse after treatment of S salbutamol in comparing of untreated DSS mice. In particular, the body weights were lower, the fecal occult blood were more severe at day 14^{th} and 22^{nd}, the length of colon-rectors was shorter in S-salbutamol and S-bambuterol treated DSS induce IBD mice than untreated DSS induced IBD mice. Histologically, inflammatory infiltration was seen in mucosa, lamina propria, submucosa and even in muscular layer. There was disappearance of crypts, degeneration of epithelia and erosion of mucosa. The histological score in both S-salbutamol and S-bambuterol treated DSS mice were markedly higher than untreated DSS mice.

This invention disclosed that, unlike their R-enantiomers, S-salbutamol and S-bambuterol were toxic rather than beneficial to the intestine of IBD. S-salbutamol and S-bambuterol treatment further exacerbate the clinic symptoms and inflammatory reaction in IBD. Furthermore, to remove S-enantiomer salbutamol and S-enantiomer bambuterol from their racemic forms, e.g. to use R-salbutamol instead of use racemic salbutamol and bambuterol can significantly reduce the toxicity and adverse effects. Therefore, this invention disclosed a new use of either R-salbutamol or R-bambuterol or other R-enantiomer β2 agonist for treatment of sub-acute or chronic IBD for reduced adverse effects. This disclosure by this invention should be considered as novel and involves an inventive step, since these toxic effects were unexpected and cannot be anticipated by a person in art.

In another embodiment, other S-enantiomers of β2 adrenergic agonist including S-terbutaline and S-clenbuterol were used in the same fashion as mentioned above in DSS mice. The similar toxic effects were observed. Both disease active index (DAI) and histological score in S-terbutaline and s-clenbuterol treated DSS mice were much worse than untreated DSS mice. This invention disclosed that S-terbutaline and S-clenbuterol were toxic to the intestine rather than therapeutic as did by their R-enantiomers.

Therefore, this invention disclosed a new use of either R-terbutaline or R-clenbuterol and other of R-enantiomers β2 agonists for treatment of subacute or chronic IBD for reduced adverse effects. The other R-enantiomers β2 agonists according to this invention includes: R or R′R′ enantiomers of short-acting β2 agonists: bitolterol, fenoterol, isoprenaline, orciprenaline or metaproterenol pirbuterol, procaterol, ritodrine; and Long-acting β2 agonists: salmeterol, tranteniterol arformoterol, Formoterol, Perforomist, salmeterol, trantinterol; and ultra-long-acting β2 agonists: abediterol, carmoterol, indacaterol, Olodaterol, vilanterol, isoxsuprine, mabuterol and zilpaterol.

In one embodiment, this invention disclosed that the therapeutic mechanism of R-enantiomers β2 agonists in IBD and its related symptoms involved in inhibition of macrophage polarization, and in inhibition of the activation of intestinal innate lymphocytes, CD4 and CD8 T lymphocytes, and induced a metabolic reprograming in these cells. On the hand, S-enantiomer β2 agonists played the opposite effects. These disclosure are novel and have not been reported in prior art.

Extra intestinal manifestations (EIMs) of inflammatory bowel disease (IBD) are common in both ulcerative colitis (UC) and Crohn's disease (CD). These manifestations can involve nearly any organ system including the musculoskeletal, dermatologic, hepatopancreatobiliary, ocular, renal, and pulmonary systems. These can cause a significant challenge to physicians in managing IBD patients. It is showed that 31.4% of UC patients and 40.4% of CD patients had 1 of the 5 major manifestations; a smaller percentage of patients had more than 1 major EIMs. Limited data have shown that approximately one third of patients will develop symptomatic primary sclerosing cholangitis (PSC) prior to a diagnosis of IBD. Based on several small studies, 10-30% of patients with arthritis related to IBD will have arthritic symptoms prior to IBD diagnosis. (Levine et al., Extraintestinal Manifestations of Inflammatory Bowel Disease. Gastroenterology & Hepatology Volume 7, Issue 4, 235 April. 2011).

Skin issues affect about 15% of all people with different types of IBD. The skin issues involve both inflammatory skin diseases (ISDs) including mainly psoriasis, rosacea, and atopic dermatitis etc. (Kim et. al., Inflammatory bowel disease is associated with an increased risk of inflammatory skin diseases, J Am Acad Dermatol. 2017 January; 76(1)); and autoimmune skin diseases including lupus, vitiligo and alopecia areata. The IBD related skin diseases also including: miliaria, pyoderma gangrenosum, Sweet's syndrome, Bowel-associated dermatosis-arthritis syndrome (BADAS), pyodermatitis-pyostomatitis vegetans (PPV). hypersensitivity vasculitis, acne, and hives (Elaine K. Luo, and Ana Gotter, 10 Skin Rashes Linked to Ulcerative Colitis, Medically reviewed Jul. 5, 2017)

This invention disclosed that R-enantiomer of β2 agonist could be used for treatment of extra intestinal manifestations of Inflammatory Bowel Disease (EMI) including arthritis, osteoarthritis, lupus, ankylosing spondylitis. vitiligo, psoriasis, amyloidosis, erythema nodosum and pyoderma gangrenosum and primary sclerosing cholangitis, acne, eczema, hives (urticarial), uveitis, iritis, episcleritis, scleromalacia, corneal ulcers, retinal vascular disease, rhinitis and pulmonary inflammatory diseases.

In one embodiment, in DSS induced IBD mice, there was also a damage of knee joints. The articular cartilage structure became thinner and partially destroyed. The chondrocytes were degenerated, decreased and clustered. The meniscuses were damaged and lost its structural integrity. In X-ray, the layer of articular cartilage was thinner and bone structure was decreased. In R-salbutamol or R-bambuterol treated mice, the damages above were significantly ameliorated. The articular cartilage and bone structure increased toward normal.

This invention provided a new use of R-salbutamol or R-bambuterol in treatment of arthritis in IBD. In other embodiment, S-salbutamol and S-bambuterol were used in DSS induced IBD mice, the damage of articular cartilage and meniscus were exacerbated comparing to untreated DSS mice. This invention disclosed that S-salbutamol and S-bambuterol were harmful to the cartilage and meniscus. Furthermore, this invention disclosed a new use of R-salbutamol or R-bambuterol for treatment of arthritic with reduced adverse effects. It is known in prior art, extra intestinal manifestations (EIMs) of IBD shares a common disease mechanism. This invention also disclosed a use of R-salbutamol or R-bambuterol for treatments of other extra-intestinal manifestations (EIMs) of IBD mentioned above, including arthritis, ankylosing spondylitis. oral Crohn's disease, lupus, amyloidosis, erythema nodosum and primary sclerosing cholangitis, eye diseases, rhinitis and pulmonary inflammation.

In an embodiment, topical used of R-salbutamol, R-terbutaline and R-clenbutarol can significantly alleviated the symptoms of acne, maliaria, hives and eczema. In rats, oral administration of above drug can significantly ameliorate psoriasis and eczema.

This invention provided a new use of R-salbutamol, R-terbutaline, R-bambuterol, R-clenbuterol in treatment of inflammatory skin diseases (ISDs) and autoimmune skin disease related to IBD including mainly: psoriasis, rosacea, atopic dermatitis, miliaria, acne, hives, pyoderma gangrenosum, Sweet's syndrome, Bowel-associated dermatosis-arthritis syndrome (BADAS), pyodermatitis-pyostomatitis vegetans (PPV). hypersensitivity vasculitis, vitiligo and alopecia areata. Other R-enantiomer adrenergic β2 agonists have similar pharmacological function. This invention disclosed use of other R-enantiomer adrenergic β2 agonists for treatment of arthritis and for treatment of other extra-intestinal manifestations (EIMs) of IBD mentioned above. The above noted other chiral β2 agonists according to this invention include R or R′R′ enantiomers of Short-acting β2 agonists: bitolterol, fenoterol, isoprenaline, orciprenaline or metaproterenol pirbuterol, procaterol, ritodrine; and Long-acting β2 agonists: arformoterol, Formoterol, Perforomist, salmeterol, trantinterol and Ultra-long-acting β2 agonists: abediterol, carmoterol, indacaterol, Olodaterol, vilanterol, isoxsuprine, mabuterol and zilpaterol.

This invention provided a new use for R-enantiomer adrenergic β2 agonist for treatment of IBD and EIMs of IBD with reduce adverse effects. This disclosure in the invention had never been reported in prior art, and it cannot be anticipated by a person in art.

The current therapy for IBD involved anti-inflammatory drugs typically, 5-aminosalicylates. Examples of this type of medication include sulfasalazine, mesalamine, balsalazide and olsalazine. On the other hand, corticosteroids can be used for more moderate or severe ulcerative colitis, which include prednisone and hydrocortisone and others. Other class of drugs for treatment of IBD involved Immunosuppressant drugs which include azathioprine and mercaptopurine, and less commonly cyclosporine. Antibodies were also used including: Infliximab, adalimumab and golimumab, and vedolizumab, certolizumab, natalizumab, Ustekinumab and Bm-ca. However, the above drugs often have severe side effects and most of them are not suitable for long-term use. The chiral R-enantiomer β2 agonists have proved to be less toxic and relatively safer in both pre-clinic and clinic studies. This invention disclosed a use of R-salbutamol or R-bambuterolorone of other R- or R′R′ enantiomer β2 agonists in combination of any one of the anti-inflammatory drugs such as 5-aminosalicylates or corticosteroid for synergistic effects or for minimize dose of marketed anti inflammatory drugs and for reduced toxicity.

This invention also disclosed a new use of R-salbutamol or R-bambuterol or one of other R- or R′R′ enantiomer β2 agonists in combination of any one of the Immunosuppressant drugs such as azathioprine and mercaptopurine for synergistic effects or for minimize the use of the dose of marketed Immunosuppressant drugs and for reduced toxicity. This invention disclosed a combination use of at least one R or R′ R-enantiomer β2 agonists with at least one of anti-inflammation medicine including at least one of 5-animoacytalates and corticosteroids, or with at least one of immunosuppression agents mentioned above, or with at least one of immune-regulatory antibodies mentioned above, or with at least one antibiotics for treatment of IBD and extra intestinal manifestation of IBD.

Sphingosine-1-phosphate (S1P) receptors is a drug target in IBD. S1P modulates lymphocyte egress from lymph nodes into the circulation and subsequently intensify an inflammatory response in the intestine by producing proinflammatory cytokines. This invention disclosed a combination use of at least one R or R′ R-enantiomer β2 agonists with at least one of S1P receptor modulators for treatment of IBD and extra intestinal manifestation of IBD.

The corticosteroid according to this invention is selected from the group consisting of budesonide, fluticasone, flunisolide, ciclesonide, mometasone, beclomethasone, and dexamethasone.

The antibiotics according to this invention are antibiotic selected from the group consisting of an aminoglycoside, an ansamycin, a carbacephem, a carbapenem, a cephalosporin, a glycopeptide, a lincosamide, a lipopeptide, a macrolide, a monobactam, a nitrofuran, a oxazolidonone, a penicillin, a polypeptide, a quinolone, a sulfonamide, a tetracycline, a chloramphenicol, a phosphonic acid antibiotic and a mycobacteria antibiotic.

The antibodies according to this invention are antibodies selected from the group consisting of Infliximab, adalimumab, golimumab, vedolizumab, certolizumab, natalizumab, ustekinumab and Bm-ca.

The S1P receptor modulators according to this invention are fingolimod, ponesimod, siponimod, ozanimod, amiselimod and GSK2018682.

In an embodiment in this invention provides a novel pharmaceutical composition comprising effective amount of R-salbutamol or R-bambuterol or other R or R′R-enantiomer of β2 agonists and their salts for used in single or in combination therapy for administration by oral, oral thin films, inhale, nasal spray, injection, topical, eye drop, rectal or vaginal administration to a patient in need. The dosage forms are solid from, solutions, nebulizer aerosol, injectable form, ointment, skin patch, thin films, soft capsule and suppository.

The enantiomer excess value or the optic purity of a R-enantiomers of β2 agonists according to this invention is no less than 80% or preferably is greater than 98% or more preferably is greater than 99%.

The pharmaceutical acceptable salts of R-enantiomers of β2 agonists according to this invention include those formed with conventional pharmaceutical acceptable inorganic or organic acids for example: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

### EXAMPLES

### Example 1

Effects of R- and S-Enantiomer β2 Agonists on DSS Induced Acute Colitis

Methods

### Animals and Drug Treatments

Male C57BL mice (8-10 weeks, 18-20 g) were grouped into control (drinking water only), DSS and DSS plus treatment groups (n=5). Acute colitis was induced by feeding mice with 2% (w/v) dextran sulfate sodium (DSS) (molecular mass 36-50 kDa) added to the drinking water given ad libitum for 7 days. At the end of seventh day, DSS was removed and animals received only drinking water for 2 days. The animals were euthanized for anatomical and histological examination at the tenth day after DSS ingestion.

R-salbutamol (R-S) (1 mg/kg, S-salbutamol (S-S) (1 mg/kg), R-terbutaline (R-T) (1 mg/kg), S-terbutaline (S-T) (1 mg/kg), R-bambuterol (10 mg/kg), S-bambuterol (S-B) (10 mg/kg), R-clenbuterol (R-C) (1 mg/kg), RS-clenbuterol (RS-C) (2 mg/kg) were administered i.g. daily, starting from the 2nd day of DSS induction for consecutive 8 days. Dexamethasone (Dex), 1 mg/kg) was used as a reference drug. The enantiomer excess value is greater than 98%, the chemical purity is greater than 99% for each of the chiral drugs.

Evaluation of disease symptoms and histological changes Disease activity index (DAI): Animals were monitored daily for weight, water/food consumption, morbidity, stool consistency and fecal blood. Disease activity index (DAI) ranging from 0 to 12 was calculated by combining each scores of weights loss, stool consistency and fecal occult blood⋅ Scores were defined as follows: for stool, stool consistency was graded 0 for no diarrhea, 2 for loose stool that did not stick to the anus, and 4 for liquid stool that stuck to the anus; for stool occult blood, presence of fecal occult blood was graded 0 for none, 2 for moderate, and 4 for gross bleeding; for weight loss, a value of 0 was assigned if the body weight remained within 1% or higher of baseline, 1 for a 1-5% loss, 2 for a 5-10% loss, 3 for a 10-15% loss, and 4 for greater than 15% loss. The colon length was recorded as an indicator of inflammation. The entire colon was cut open longitudinally and gently flushed with sterile phosphate-buffered saline (PBS) to remove any traces of feces for pathology preparation. Colon segments were fixed in 10% neutral buffered formalin. Tissues were embedded in paraffin, sectioned, mounted on slides. H&E-stained paraffin section images were acquired. Pathological evaluation was conducted in a blinded manner.

Histological Score: the scores were given as follows: epithelium, 0=normal morphology, 1=loss of goblet cells, 2=loss of goblet cells in large areas, 3=loss of crypts, and 4=loss of crypts in large areas; and inflammation cells infiltration, 0=no infiltrate, 1=infiltrate around the crypt basis, 2=infiltrate reaching the muscularis mucosae, 3=extensive infiltration reaching the muscularis mucosae and thickening of the mucosa with abundant edema, and 4=infiltration of the submucosa. Data were presented as mean±the standard deviation (SD).

### Results

### β2-Receptor Agonist Ameliorated DSS-Induced Acute Colitis.

Mice showed symptoms of colitis, including weight loss, diarrhea, fecal blood after 7 days orally administered of 2% DSS. The disease active index (DAI) value was significantly increased in DSS mice. The treatments of β2-receptor agonists significantly ameliorated the symptoms and reduce the DAI values and the histological score. The therapeutic effects of R-enantiomer of β2-receptor agonists were better than the reference drug dexamethasone. (Table 1)

TABLE 1
Effects of different treatments in DSS induce acute colitis

| | Con | DSS | R-S | S-S | R-T | S-T | R-B | S-B | R-C | RS-C | DEX |
|---|---|---|---|---|---|---|---|---|---|---|---|
| DAI | 0 | 10 | 4 | 13 | 8 | 12 | 6 | 12 | 5 | 10 | 7 |
| Weight | 105 ± 2.6 | 85 ± 0.6 | 93 ± 2.8 | 80 ± 2.3 | 88 ± 1.7 | 80 ± 2.9 | 92 ± 3.3 | 81 ± 3.2 | 89 ± 2.0 | 87 ± 2 | 88 ± 2.4 |
| FOB | 0.5 ± 0 | 4 ± 0.5 | 0.5 ± 0.5 | 4.5 ± 0.5 | 2 ± 0 | 4 ± 0.5 | 1 ± 0.5 | 2.5 ± 0.5 | 2 ± 0.5 | 4 ± 0.1 | 2.5 ± 0.5 |
| Col | 8.6 ± 0 | 6.1 ± 0.3 | 7.7 ± 0.6 | 6.0 ± 0.9 | 6.6 ± 0.3 | 5.9 ± 0.5 | 7.2 ± 0.2 | 6 ± 0.6 | 7 ± 0.7 | 6.5 ± 0.2 | 7.1 ± 0.2 |
| Length | | | | | | | | | | | |
| Hist | 0 | 7.0 ± 0.5 | 4 ± 0.5 | 7.9 ± 0.5 | 6.0 ± 1 | 7 ± 1 | 5 ± 0.5 | 7.5 ± 1 | 4.5 ± 0.5 | 6 ± 0.5 | 6 ± 0.5 |
| score | | | | | | | | | | | |

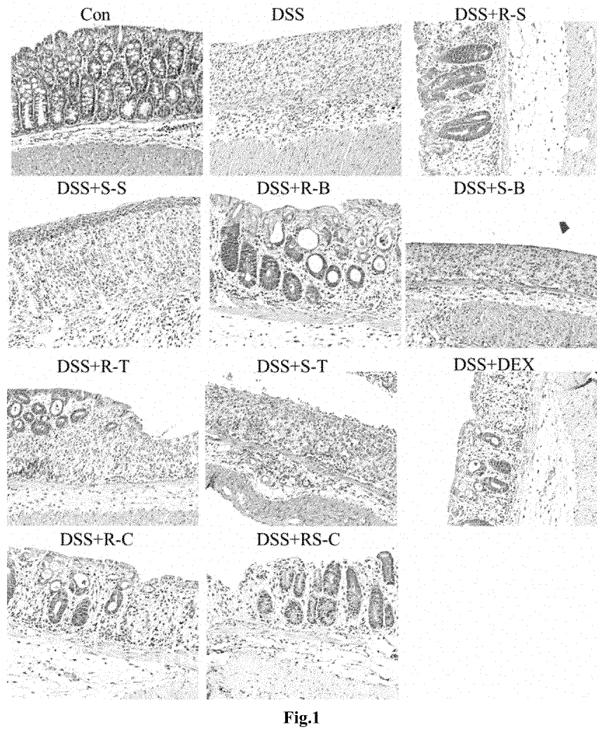

DAI: disease active index, FOB: fecal occult blood, Col.: Colon, Hist: histological.
FIG. 1. DSS induced Colitis and treatment with RS (R-salbutamol), RB (R-bambuterol), RT (R-terbutaline), RC (R-clenbuterol), S-S(S-salbutamol), SB (S-bambuterol), S-T(S-terbutaline), RS-C (Racemic clenbuterol) and DEX (Dexamethasone) for one week.

Histological analysis of H&E-stained colon specimens from the control group showed that colonic mucosal epithelium retained its integrity and was continuous, the crypts were arranged regularly with clear structure, and there was no inflammatory cell infiltration and ulceration. In the DSS model group, the mucosa lost its architecture, the mucosal epithelium was discontinuous, extensive ulcer lesions formed, disappearance of crypts, destruction of lamina propria, inflammation extended through mucosa and submucosa, and massive inflammatory cell infiltration was present. In the R-enantiomer treated groups (DSS+R-A or R-B or R-T or R-C), the histological integrity was greatly improved, and the lamina propria was intact.

The crypts were regenerated, clear recovery of mucosa epithelia, and significant less inflammatory infiltration. In DSS+DEX treated animal, Inflammation infiltration was seen in mucosa and submucosa and the lamina propria was destructed, although there were a few incomplete growing crypts and mucosa epithelia. In general, there was some improvements with DSS+DEX treatment, but it was less in comparing treatment of R-enantiomer β2 agonists.

In S-enantiomer treated DSS mice (DSS+S-A or S-B or S-T), there were no signs of improvements in comparing untreated DSS mice. In the contrary, the inflammatory infiltration was more server in mucosa, submucosa and muscular layer (DSS+S-B), and lamina propria were completely destructed (DSS+S-A). There was a significant execration of pathological histology. The histological score in S-enantiomers treated DSS mice was higher than untreated DSS mice and much higher than R-enantiomers treated DSS mice.

When a racemic was used as in RS-Clenbuterol, the historical score was better than DSS untreated but worse than R-enantiomer treated mice. In DSS mice treated with RS clenbuterol, there was disappearance of mucosa epithelia, destruction of lamina propria and inflammatory infiltration in mucosa, submucosa and muscular layer, although a few crypts were regenerated. The overall histology and DAI in RS clenbuterol mice showed much less improvement than DSS mice treated with R-clenbuterol.

### Example 2

Effects of R- and S-Enantiomer β2 Agonists on DSS Induced Sub-Acute Colitis

### Methods

Same as example 1, except
1), Three weeks' study with one week treatment.

DSS and all the treatment was removed and replaced with drinking water at the end of 1^{st} week. Mice were given drink water and food ad libitum for additional 2 weeks. The evaluation and examination were made as did in example 1 at 23^{rd} day.
2), Only R-salbutamol (R-S) (1 mg/kg), S-salbutamol (S-S) (1 mg/kg), R-bambuterol (10 mg/kg), S-bambuterol (S-B) (10 mg/kg) were used.

### Results

Effects of R-bambuterol on disease active index and histological score.
In DSS induce IBD mice, one week after withdrew of DSS, there were still significant loss of weight and fecal occult blood at day 14th and 22nd although there was a slightly improvement comparing the days 7th. The DAI were increased markedly. The disease active index (DAI) was significant improved after the R-salbutamol treatment in comparing of untreated DSS animals. There body weights of mice similar as control normal mice, the fecal occult blood was significantly reduced after the treatment of R-salbutamol and R-bambuterol in comparing to DSS mice. (table 2)

In mice with DSS induced colitis, the colon-rector length was significant shortened and histological score were significant higher from control. There were significant mononuclear leucocytes infiltration; few newly grew crypts with architectural disarray, denegation of epithelia, mucosa aberrant and destruction of lamina propria and formation of granuloma. The histological index was also significantly ameliorated after R-salbutamol and R-bambuterol administration. There were completed regeneration and normal arranged crypts and normal grew epithelia in mucosa. The lamina propria appeared normal. There were few inflammatory infiltrations in either mucosa or submucosa.

TABLE 2
Effects of different treatments in DSS colitis

| | Con | DSS | R-S | S-S | R-B | S-B |
|---|---|---|---|---|---|---|
| DAI | 0 | 10 | 4 | 13 | 6 | 12 |
| Weight | 118 ± 1.3 | 100 ± 0.4 | 114 ± 0.2 | 95 ± 0.8 | 112 ± 0.6 | 96 ± 1.1 |
| FOB | 0 ± 0.5 | 3 ± 0.5 | 0.5 ± 0.5 | 4.5 ± 0.3 | 1.1 ± 0.5 | 3.9 ± 0.1 |
| Col Length | 9.3 ± 0.5 | 7.1 ± 0.4 | 8.2 ± 0.6 | 6.5 ± 0.9 | 7.9 ± 0.3 | 6.8 ± 0.1 |
| Hist Score | 0 | 6.4 ± 0.5 | 3 ± 0.5 | 7.7 ± 0.5 | 4 ± 1 | 6.9 ± 1 |

DAI. disease active index,
FOB. fecal occult blood,
Col. Colon,
Hist. histological

### Example 3

Effects of Prolonged Treatments in DSS Induced Sub-Acute Colitis

### Methods

Same as example 2, except.
1), Two Weeks Treatment within a Three Weeks of Study

DSS was removed from drinking water at the end of 1st week. The treatments were started at 1^{st} day for consecutive 14 days (2 weeks). Only drinking water given for the 3rd week. The evaluation and examination were made at 23rd day as did in example 1. Treatment includes R-salbutamol (R-S) (1 mg/kg), S-salbutamol (S-S) (1 mg/kg), R-bambuterol (10 mg/kg), S-bambuterol (S-B) (10 mg/kg).

### Results

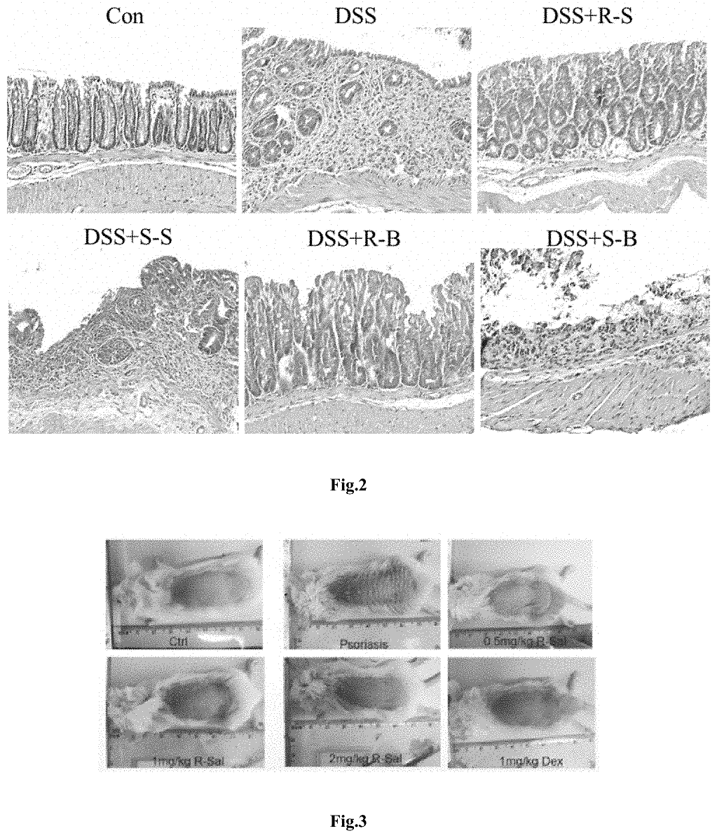

The DAI and histological score were significantly higher in untreated DSS mice, although there were signs of recovery from the acute phase. Both the DAI and histological score were return toward normal in DSS mice treated with R-salbutamol (DSS+R-S) or R-bambuterol (DSS+R-B). There was fully grown mucosa, epithelia and crypts with normal arrangements and architectures. Mucosa, submucosa and lamina propria were normal without inflammatory infiltration after two weeks treatment of R-bambuterol or R-salbutamol. (FIG. 2, DSS+R-B and DSS+R-S). On the contrary, in DSS mice treated with S-salbutamol and S-bambuterol, there was an exacerbation of symptoms of colitis. Both DAI and histological score were markedly higher than untreated DSS group. There were ulceration and lesions in mucosa epithelia, the crypts architecture was disarrayed, lamina propria and muscular were destructed. Inflammatory infiltration was seen in all layers and dysplasia lesions. (FIG. 2, DSS+S-S, DSS+S-B) In general, in DSS induced chronic colitis, treatment with S-salbutamol and S-bambuterol can cause serious exacerbation of the diseases in comparison of untreated.

FIG. 2. DSS induced Colitis and treatment with R-S (R-salbutamol), S-S (S-salbutamol), R-B (R-bambuterol) and S-B (S-bambuterol) for two week.

### Example 4

Effects of R-Salbutamol on Psoriasis, a EIMs of IBD

Psoriasis is one of the main extra intestinal manifestations (EIMs) of IBD. Since psoriasis occurs only in a portion of the DSS or TNBS induced IBD mice in this invention, a well accepted psoriasis mice model, IMQ-induced Psoriasis-Like Skin lesions was then used for better control in the follow study.

### Method:

Mice were divided into control, psoriasis and psoriasis plus treatment groups, (n=8 in each group). Psoriasis were induced by topical application of imiquimod cream (62.5 mg) on shaved dorsal skin for seven consecutive days. Vaseline was used in control group. There were four treatment groups: three doses of R-salbutamol (R-Sal) (0.5 mg, 1 mg, 2 mg/kg) and Dexamethasone (1 mg/kg) as positive control. All the treatments and same volume of saline (in control group) were administered orally. In some experiment, R-bambuterol (long-last beta2 agonist) was used as an additional treatment group (n=3). Psoriasis Area Severity Index were used for evaluation. Each of the scores for erythema, scaling and skin thickness were measured separately and then group together as a calculative score. (FIG. 2)

### Result

1). Skin Manifestation and Psoriasis Area Severity Score.

Application of imiquimod cream induced a typical psoriasis lesions. There was a significantly improvement of the lesions after drug treatments. (FIG. 1) The Psoriasis Area Severity score was the highest in Psoriosis (Pso) group. The scores were significantly reduced after treatments. There was a dose-dependent response to R-salbutamol. The high dose of R-salbutamol (2.0 mg/kg) showed the best effects for all the scores and the results is similar to the effects of dexamethasone (Dex) (FIG. 2).

FIG. 1. The effect of R-salbutamol (R-Sal) and dexamethasone (Dex) on IMQ-induced psoriasis-like dorsal skin lesions. The photos were taken on day 8th.
FIG. 2. The Psoriasis Area Severity score of IMQ-induced psoriasis-like dorsal skin lesions after treatments of dexamethasone (Dex) and R-salbutamol of 0.5 mg/kg (R1), 1.0 mg/kg (R2), and 2.0 mg/kg (R3) respectively.

2). Changes of Histology

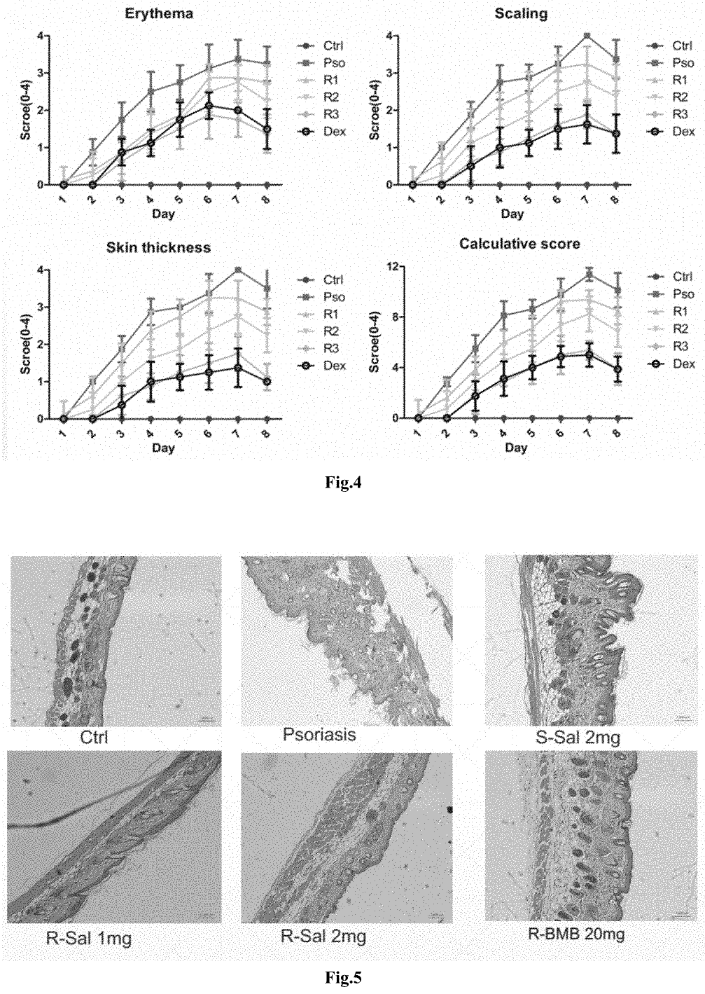

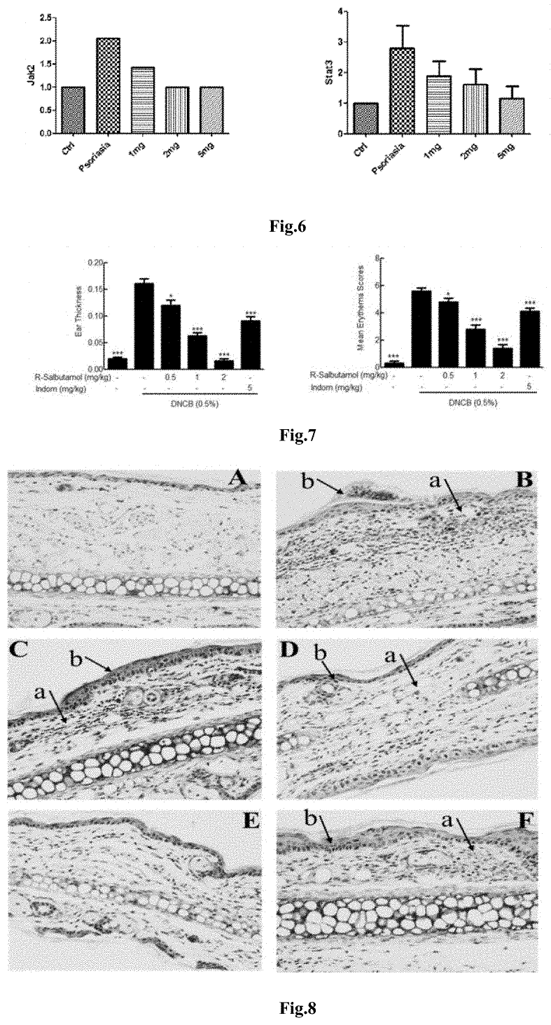

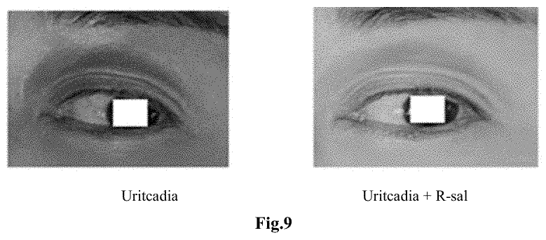

Histological changes in psoriasis and after treatment of R-Salbutamol and R-bambuterol and S-salbutamol disclosed as FIG. 3. The psoriasis skin lesions were severe after topical application of IMQ. The lesion was mostly recovered after treatments of both R-salbutamol and R-bambuterol. Surprisingly, S-salbutamol, the S enantiomer of R-salbutamol, worsened the psoriasis histology. There were widened lesion, epidermal hyperplasia and thickening (acanthosis), elongation of rete ridges and super-dermis/dermis inflammatory infiltrations. The worsening effects of S-salbutamol on psoriasis has never been disclosed in prior art.

FIG. 3. The HE stains of dorsal skin in IMQ-induced psoriasis mice before and after treatments of R-salbutamol (R-Sal), R-Bambuterol (R-BMB) and S-salbutamol. Ctrl. control.

3) Signal Pass-Way

There was enhanced expression of mRNA of Janus kinase 2 (JAK2), a non-receptor tyrosine kinase, and the signal transducer and activator of transcription protein (Stat 2) in the plasma of psoriasis mice in comparison of control, Indicating an increase of JAK2 and Stat 2 proteins. These over expression of mRNA was inhibited by R-salbutamol in a dose-response fashion. (FIG. 3 left & right)

This invention disclosed that the therapeutic effects of R-salbutamol, R-β2 agonist, on psoriasis involves Jak2 and Stat2 signal pass way. This particular signal pass way is also known to be involved in IBD. This disclosed of this invention is novel and cannot be anticipated by a person in art.

### Example 5

Effects of R-Salbutamol on Eczema, an EIM of IBD

Eczema (Atopica dermatitis) is one of the main extra intestinal manifestations (EIMs) of IBD. Since it occurs only in a portion of the DSS or TNBS induced IBD mice in this invention, a commonly accepted Atopica dermatitis mice model, 2, 4-dinitrochlorobenzene (DNCB) induced allergic contact dermatitis, was used in the follow study. (Ku et al. The prevention of 2,4-dinitrochlorobenzene-induced inflammation in atopic dermatitis-like skin lesions in BALB/c mice by Jawoongo, BMC Complementary and Alternative Medicine (2018) 18:215)

### Methods:

DNCB Induced Allergic Contact Dermatitis Model (Eczema)

Mice was shaved at abdominal area DNCB (100 μL 0.5%) is dissolved and applied to the shaved skin to induce Atopica dermatitis for 2 consecutive days. At day 8^{th}, DNCB was applied to both inner and outer surfaces of ears to induce eczema. Treatment of R-salbutamol and indomethacin were given orally at day 1^{st} for consecutive 8 days. The mice were scarified at day 9^{th} for examination and evaluation.

### Anima and Treatments

Mice were divided into four groups (n=8), namely A, Control; B. Eczema (DNCB); C. Eczema plus R-Salbutamol of 0.05, 0.1 or 0.2 mg/kg; D. Eczema plus indomethacin of 5 mg/kg. The inflammation scores were evaluated according to swelling, erythema, scratch and thickness of both ears.

The Score standard

| item | intensity | score |
|---|---|---|
| erythema | absent | 0 |
| | Tiny pinkish | 1 |
| | Moderate red color without scad | 2 |
| | Moderate severe red color with scad | 3 |
| | Severe red color with severe scad | 4 |
| swelling | Absent | 0 |
| | Mild swelling | 1 |
| | Moderate swelling | 2 |
| | Severe swelling | 3 |
| Scratch | Absent | 0 |
| | Existed | 1 |

### Results:

1), Changes of Inflammation Score

The thickness and erythema scores of both ears, as an inflammatory index of eczema, were significantly increased in eczema in comparing of control. Both of the thickness and erythema were significantly alleviated or prevented after treatments of various dose of R-salbutamol. However, indomethacin, the well-known cox-inhibitor anti-inflammatory agent, showed lesser effects.

2) Changes of Histology

DNCB induced a severe eczema, R-salbutamol significantly improved eczema histology and reduced the inflammatory infiltration with a dose-dependent response. However, indomethacin showed little effects on eczema. (Fig A-F)

Figure. Effects of R-Salbutamol on ear skin histopathology in mice treated with DNCB (magnification 400×). (A) control. (B) eczema: monocyte infiltration (a) and epidermal keratinization (b). (C) R-Salbutamol (0.05 mg/kg): little effects on eczema (D) R-Salbutamol (0.1 mg/kg): significant reduced monocyte infiltration. (E) R-salbutamol (0.2 mg/kg): further reductions in monocyte infiltration and epidermal keratinization. (F) Indomethacin (5 mg/kg/day).

### Example 6

Effects of R-Salbutamol on Hives (Uritcadia), a EIM of IBD

A health female with uritcadia on her face and eyelid area and it had been lasted for a week. R-salbutamol (R-sal) cream (5%) was applied topically overnight. The symptom was significantly alleviated at the morning of the second day. (Figure below).
Uritcadia Uritcadia+R-sal

1, Methods of use optically pure R or R′R-enantiomer of adrenergic β2 receptor agonists as well as their pharmaceutically suitable salts in a pharmaceutical composition or in combination with anti-inflammation or immunosuppression medicine or antibodies or antibiotics or S1P receptor modulators in the manufacture of a medicament for prevention or treatment of inflammation bowel diseases and extra intestinal manifestation of inflammation bowel disease to a patient in need.

2, The said R or R′R-enantiomer of adrenergic β2 receptor agonists according to claim 1, are short acting β2 agonist including: salbutamol, terbutaline, bitolterol, fenoterol, isoprenaline, orciprenaline or metaproterenol pirbuterol, procaterol and ritodrine.

3, The said R or R′R-enantiomer of adrenergic β2 receptor agonists according to claim 1, are long-acting β2 agonists: bambuterol, arformoterol, formoterol, perforomist, salmeterol, trantinterol and Ultra-long acting β2 agonists: abediterol, carmoterol, indacaterol, olodaterol, vilanterol, isoxsuprine, mabuterol and zilpaterol.

4, The optically pure R-enantiomers according to claim 1, are of an enantiomer excess value greater than 80%.

5, The optically pure according to claim 1, are of an enantiomer excess value greater than 98.5% and preferably greater than 99%.

6, The said inflammation bowel disease according to claim 1, are Crohn's disease and ulcerative colitis.

7, The said Crohn's disease in claim 6, including perianal symptoms include perianal erythema, abscesses, ulcers and perianal fissures or fistulas.

8, The said perianal symptoms according to claim 7, include hemorrhoid inflammation symptoms.

9, the said Crohn's disease in claim 6 including fibrosis and intestinal lumen narrowing.

10, The said extraintestinal manifestations according to claim 1, are arthritis, osteoarthritis and ankylosing spondylitis.

11, The said extraintestinal manifestations according to claim 1, are rhinitis and uveitis.

13, The said extraintestinal manifestations according to claim 1, are oral Crohn's disease, amyloidosis, episcleritis, scleromalacia, corneal ulcers, primary sclerosing cholangitis, lupus and pulmonary inflammatory disease.

14, The said extraintestinal manifestations according to claim 1, are inflammatory skin disease including atypical dermatitis, psoriasis, rosacea, miliaria, acne, pyoderma gangrenosum, Sweet's syndrome, Bowel-associated dermatosis-arthritis syndrome (BADAS), pyodermatitis-pyostomatitis vegetans (PPV). hypersensitivity vasculitis.

15, The said extraintestinal manifestations according to claim 1, are autoimmune skin diseases including urticarial (hives), vitiligo, and alopecia areata.

16, The said treatment in claim 1, involves inhibition of Jak2 and Stat2 signal pass way.

17, The said anti-inflammation medicine according to claim 1 are 5-aminosalicylates including sulfasalazine, mesalamine, balsalazide olsalazine and corticosteroids including prednisone, budesonide, fluticasone, flunisolide, ciclesonide, mometasone, beclomethasone, and dexamethasone.

18, The said anti-inflammation medicine according to claim 1 are 5-aminosalicylates including sulfasalazine, mesalamine, balsalazide and olsalazine.

19, The said antibiotics according to claim 1, are aminoglycoside, ansamycin, carbacephem, carbapenem, cephalosporin, glycopeptide, lincosamide, lipopeptide, macrolide, monobactam, nitrofuran, oxazolidonone, penicillin, polypeptide, aquinolone, sulfonamide, tetracycline, chloramphenicol, phosphonic acid antibiotic and a mycobacteria antibiotic.

20, The said antibodies according to claim 1, are Infliximab, adalimumab, golimumab, vedolizumab, certolizumab, natalizumab, Ustekinumab and Bm-ca.

21, The said S1P receptor modulators according to claim 1, are fingolimod, ponesimod, siponimod, ozanimod, amiselimod and GSK2018682.

22, The said treatment according to claim 1, involves in inhibition of macrophage polarization. and induced a metabolic reprograming in these cells.

23, The said treatment according to claim 1, involves in inhibition of the activation of intestinal innate lymphocytes, CD4 and CD8 T lymphocytes.

24, The said treatment according to claim 1, involves in a metabolic reprograming in cells including macrophages, innate lymphocytes as well as CD4 and CD8 T lymphocytes.

25, The said pharmaceutically suitable salts according to claim 1 are those formed with conventional pharmaceutical acceptable inorganic or organic acids including: hydrochloride, hydrobromide, sulphate, hydrogen sulphate, dihydrogen phosphate, methanesulphonate, bromide, methyl sulphate, acetate, oxalate, maleate, fumarate, succinate, 2-naphthalene-sulphonate, glyconate, gluconate, citrate, tartaric, lactic, pyruvic isethionate, benzenesulphonate or para-toluenesulphonate.

26, The said pharmaceutical composition according to claim 1, is for administration by oral, inhale, nasal spray, injection, topical, eye drop, rectal or vaginal administration and is in the dosage forms of solid form, solutions, nebulizer aerosol, injectable form, ointment, skin patch, thin film, soft capsule and suppository to a patient in need.
